(19) United States
(12) Patent Application Publication
(10) Pub. No.: US 2026/0210918 A1
(43) Pub. Date: Jul. 23, 2026

(54) METHOD FOR SIMULTANEOUSLY DETECTING FOUR ALKALOIDS AND 10 CATECHIN COMPONENTS IN TEA

(71) Applicant: Tea Research Institute, Guangdong Academy of Agricultural Sciences, Guangzhou (CN)

(72) Inventors: Dandan Qin, Guangzhou (CN); Hualing Wu, Guangzhou (CN); Kaixing Fang, Guangzhou (CN); Erdong Ni, Guangzhou (CN); Qiushuang Wang, Guangzhou (CN); Qing Wang, Guangzhou (CN); Hongjian Li, Guangzhou (CN); Bo Li, Guangzhou (CN); Chendong Pan, Guangzhou (CN)

(21) Appl. No.: 19/432,386

(22) Filed: Dec. 24, 2025

(30) Foreign Application Priority Data
Jan. 17, 2025 (CN) 202510078190.7

(51) Int. Cl. G01N 30/86; G01N 30/02; G01N 30/04; G01N 30/06; G01N 30/14; G01N 30/30; G01N 30/32; G01N 30/74; G01N 30/88

(52) U.S. Cl. CPC G01N 30/8631; G01N 30/14; G01N 30/74; G01N 2030/027; G01N 2030/047; G01N 2030/062; G01N 2030/146; G01N 2030/3007; G01N 2030/324; G01N 2030/884

(57) ABSTRACT

The present disclosure belongs to the technical field of tea component detection methods, and specifically relates to a method for simultaneously detecting four alkaloids and 10 catechin components in tea. The method includes: simultaneously detecting four alkaloids and 10 catechin components in a tea sample by using HPLC, and obtaining the content of the 14 characteristic components in the tea sample according to respective standard curves of the four alkaloids and 10 catechin components. The detection method provided by the present disclosure enables simultaneous detection and separation of 14 compounds in tea, which solves the problem of poor separation effect when simultaneously analyzing multiple compounds in tea in the prior art, meets high-standard requirements for tea component analysis, provides a more efficient, accurate and reliable analysis tool for tea producers, testing institutions and researchers, and provides strong support for tea quality control, scientific research, deep processing, and other fields.

### TECHNICAL FIELD

The present disclosure belongs to the technical field of tea component detection methods, and specifically relates to a method for simultaneously detecting four alkaloids and 10 catechin components in tea.

### BACKGROUND

As a widely consumed beverage, tea contains a variety of bioactive components that have significant impacts on human health. Among the bioactive components, theophylline, theobromine, caffeine, and theacrine are common alkaloids in tea, while catechins belong to a class of polyphenolic compounds with extensive bioactivities and are the most important physiologically active substances in tea. Catechins are mainly composed of monomers such as epigallocatechin gallate (EGCG), epigallocatechin (EGC), epicatechin gallate (ECG), and epicatechin (EC). Many physiological and pharmacological functions of catechins have been verified, such as antioxidation, antimutagenesis, anticancer, anti-atherosclerosis, antibacteria, and the like. Accurate detection and separation of these compounds are of great significance for tea quality control, scientific research, deep processing, and other fields.

In the prior art, a method for detecting content of polyphenols and catechins in tea is provided in the National Standard “Determination of total polyphenols and catechins content in tea” (GB/T 8313-2018), but there exist obvious deficiencies in simultaneously detecting and separating four alkaloids and 10 catechins in tea. Many compounds cannot be effectively separated through this method, leading to inaccurate analysis results and failure to meet needs of tea quality control and scientific research. This limits the accuracy and reliability of chemical composition analysis of tea and hinders the further development of the tea industry and the in-depth scientific research.

### SUMMARY

An objective of the present disclosure is to provide a method for simultaneously detecting four alkaloids and 10 catechin components in tea, so as to solve the above problems.

According to one aspect of the present disclosure, a method for simultaneously detecting four alkaloids and 10 catechin components in tea is provided, including the following steps:

simultaneously detecting four alkaloids and 10 catechin components in a tea sample by using high-performance liquid chromatography (HPLC), and obtaining the content of the 14 characteristic components in the tea sample according to respective standard curves of the four alkaloids and 10 catechin components; chromatographic conditions are as follows: a chromatographic column is Agilent Zorbax Eclipse Plus C18, a column temperature is 30-40° C., a mobile phase C is 100% acetonitrile, a mobile phase D is a 0.05% phosphoric acid aqueous solution, a flow rate is 0.9-1.1 mL/min, and a detection wavelength is 275-285 nm; and performing quantification based on a peak area according to an external standard method, where a gradient elution program is as follows:

| Time (min) | Mobile phase C/% | Mobile phase D/% |
|---|---|---|
| 0.01 | 6.5 | 93.5 |
| 30 | 16 | 84 |
| 34 | 20 | 80 |
| 39 | 20 | 80 |
| 40 | 35 | 65 |
| 45 | 35 | 65 |
| 50 | 6.5 | 93.5 |
| 60 | End | End |

In some embodiments, the four alkaloids are theophylline, theobromine, caffeine, and theacrine; and the 10 catechins are epicatechin (EC), catechin (C), epigallocatechin (EGC), epicatechin gallate (ECG), epigallocatechin gallate (EGCG), catechin gallate (CG), gallocatechin gallate (GCG), gallocatechin (GC), epigallocatechin-3-O-(3-O-methyl) gallate, and theaflavin.

In some embodiments, standard curves are obtained by the following method: accurately weighing standard samples of catechins and alkaloids respectively, placing the samples in volumetric flasks, dissolving the samples with a methanol solution, and making up to volume to prepare a standard stock solution with content of 1 mg/mL for each standard substance; and then diluting the standard stock solution with the methanol solution to prepare a standard solution with a series of gradient concentrations of 0.0012 mg/mL, 0.002 mg/mL, 0.006 mg/mL, 0.01 mg/mL, 0.03 mg/mL, 0.05 mg/mL, and 0.2 mg/mL respectively, performing detection according to the above chromatographic conditions to obtain corresponding chromatograms, and plotting standard curves with concentration as the ordinate and peak area as the abscissa.

In some embodiments, the 0.05% phosphoric acid aqueous solution is prepared by the following method: pipetting 0.50 mL of phosphoric acid, adding water and making up to volume in a 1,000 mL volumetric flask, and mixing well, where a pH value is 1.8-2.0.

In some embodiments, a flow rate is 1 mL/min, a column temperature is 35° C., a detection wavelength is 280 nm, a chromatographic column specification is 4.6×250 mm, 5 μm, and an injection volume is 10 μL.

In some embodiments, a method for tea sample treatment is as follows: after grinding tea, adding a 70% methanol aqueous solution preheated in a water bath, fully mixing, immediately transferring a resulting mixture to the water bath for extraction, cooling to room temperature after the extraction, and centrifuging to collect a supernatant; and extracting centrifuged residues once with the 70% methanol aqueous solution, repeating the above operation, combining extracts and making up to volume, shaking well, and filtering.

In some embodiments, a method for tea sample treatment is as follows: weighing 0.2 g (accurate to 0.0001 g) of a uniformly ground tea sample, adding the sample into a 10 mL centrifuge tube, adding 5 mL of a 70% methanol aqueous solution preheated in a 60-80° C. water bath, fully mixing, immediately transferring a resulting mixture to the 60-80° C. water bath for extraction for 8-12 min, shaking every 3-6 min, cooling to room temperature after extraction, centrifuging at 3,000-4,000 r/min for 8-12 min, and transferring a supernatant to a 10 mL volumetric flask; and extracting residues once with 5 mL of a 70% methanol aqueous solution, repeating the above operation, combining extracts and making up to volume of 10 mL, shaking well, and filtering with a 0.45 μm filter membrane.

In some embodiments, a method for tea sample treatment is as follows: weighing 0.2 g (accurate to 0.0001 g) of a uniformly ground tea sample into a 10 mL centrifuge tube, adding 5 mL of a 70% methanol aqueous solution preheated in a 70° C. water bath, fully mixing, immediately transferring a resulting mixture to the 70° C. water bath for extraction for 10 min, shaking every 5 min, cooling to room temperature after the extraction, centrifuging at 3,500 r/min for 10 min, and transferring a supernatant to a 10 mL volumetric flask; and extracting residues once with 5 mL of a 70% methanol aqueous solution, repeating the above operation, combining extracts and making up to volume of 10 mL, shaking well, and filtering with a 0.45 μm filter membrane.

The detection method provided by the present disclosure enables simultaneous detection and separation of four alkaloids and 10 catechins, which solves the problem of poor separation effect when simultaneously analyzing multiple compounds in tea in the prior art, meets high-standard requirements for tea component analysis, provides a more efficient, accurate and reliable analysis tool for tea producers, testing institutions and researchers, and provides strong support for tea quality control, scientific research, deep processing, and other fields.

### BRIEF DESCRIPTION OF THE DRAWINGS

FIG. 1 is a chromatogram of standard samples of four alkaloids and 10 catechin components.

FIG. 2 is a chromatogram of four alkaloids and 10 catechin components in black tea.

FIG. 3 is a chromatogram of four alkaloids and 10 catechin components in yellow tea.

FIG. 4 is a chromatogram of four alkaloids and 10 catechin components in green tea.

FIG. 5 is a chromatogram of four alkaloids and 10 catechin components in dark tea.

FIG. 6 is a chromatogram of four alkaloids and 10 catechin components in white tea.

FIG. 7 is a chromatogram of four alkaloids and 10 catechin components in oolong tea.

FIG. 8 is a chromatogram of four alkaloids and 10 catechin components in fresh tea leaves.

FIGS. 9 to 22 are standard curve diagrams of different substances.

### DETAILED DESCRIPTIONS OF THE EMBODIMENTS

The present disclosure will be further described in detail below with reference to specific embodiments. In an embodiment, reagents used and their sources are as follows: phosphoric acid, sourced from Tianjin Fuyu Fine Chemical Co., Ltd.; standard samples of epicatechin (EC), catechin (C), epigallocatechin (EGC), epicatechin gallate (ECG), epigallocatechin gallate (EGCG), catechin gallate (CG), gallocatechin gallate (GCG), gallocatechin (GC), theophylline, epigallocatechin-3-O-(3-O-methyl) gallate (EGCG3″Me), caffeine, theobromine, theacrine, and theaflavin with purity ≥98%, sourced from Shanghai Aladdin Biochemical Technology Co., Ltd.; and all organic solvents for (liquid chromatography) mobile phases are domestic and chromatographically pure.

This embodiment mainly implements the following steps:

Treatment of tea leaves: weighing 0.2 g (accurate to 0.0001 g) of uniformly ground tea leaves into a 10 mL centrifuge tube, adding 5 mL of a 70% methanol aqueous solution preheated in a 70° C. water bath, fully mixing, immediately transferring a resulting mixture to the 70° C. water bath for extraction for 10 min, shaking every 5 min. Extracting centrifuged residues once with 5 mL of a 70% methanol aqueous solution, repeating the above operation, combining extracts, making up to volume of 10 mL in the volumetric flask, and shaking well. Storing the extracts in a −20° C. refrigerator, and filtering with a 0.45 μm filter membrane before determining catechin and alkaloid components for use.

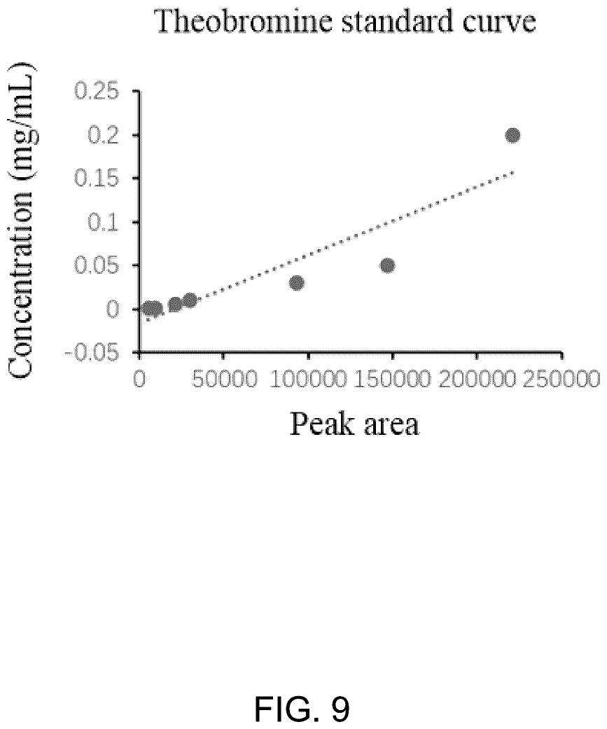

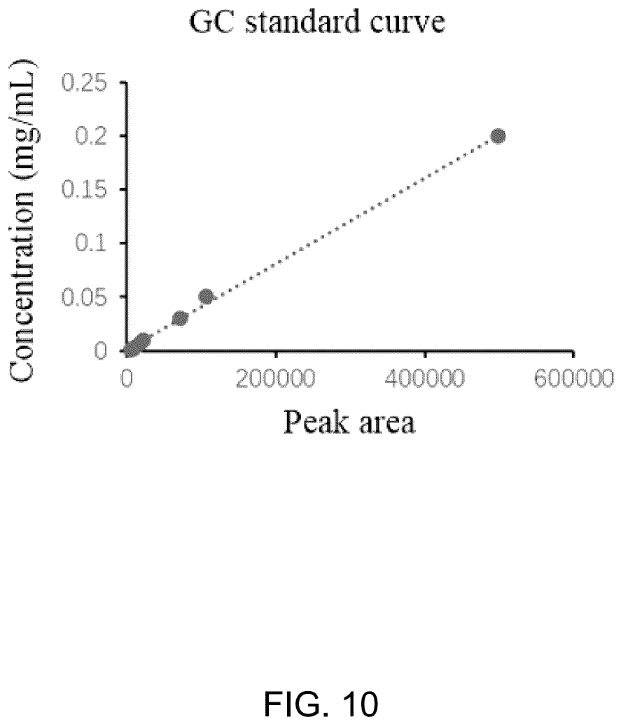

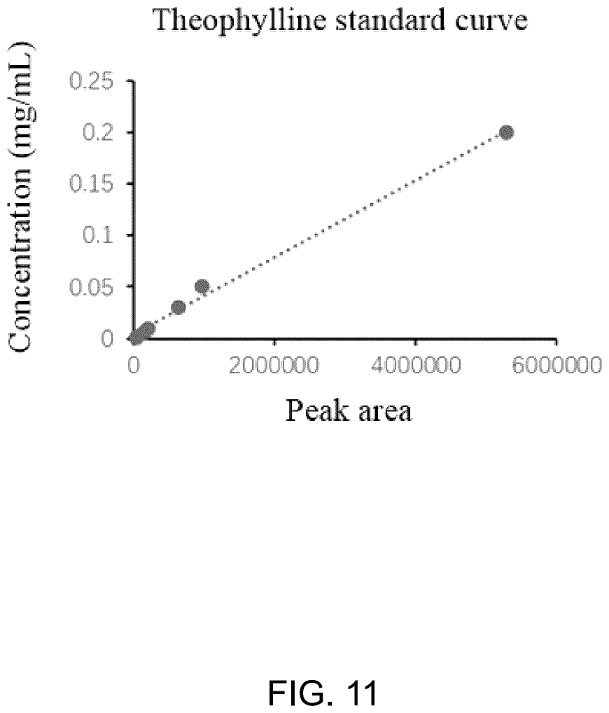

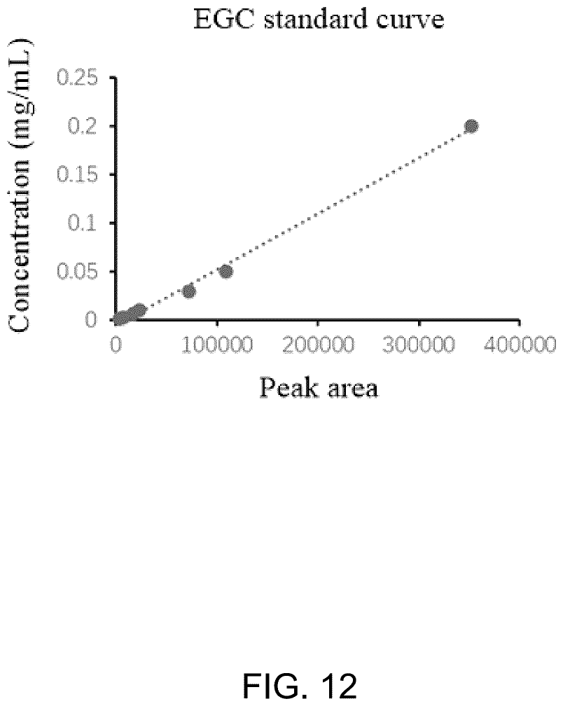

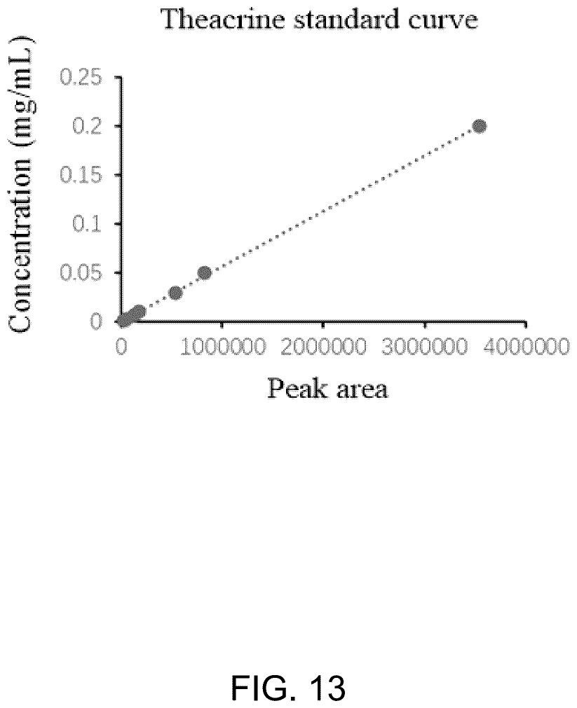

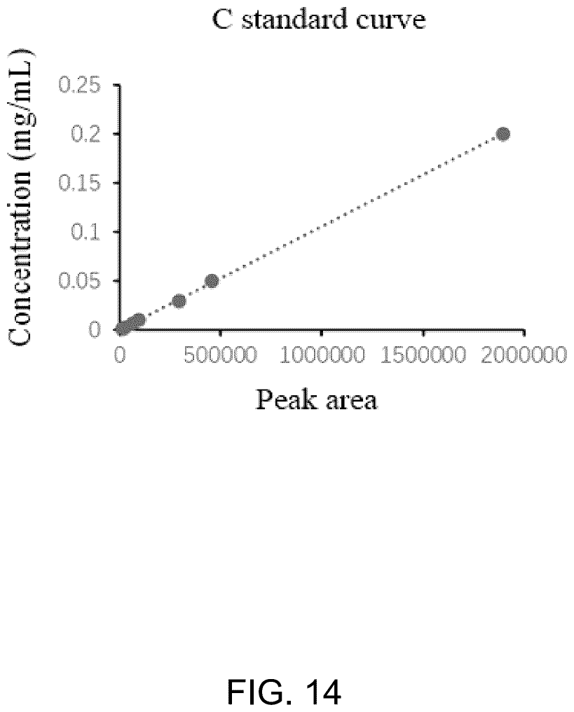

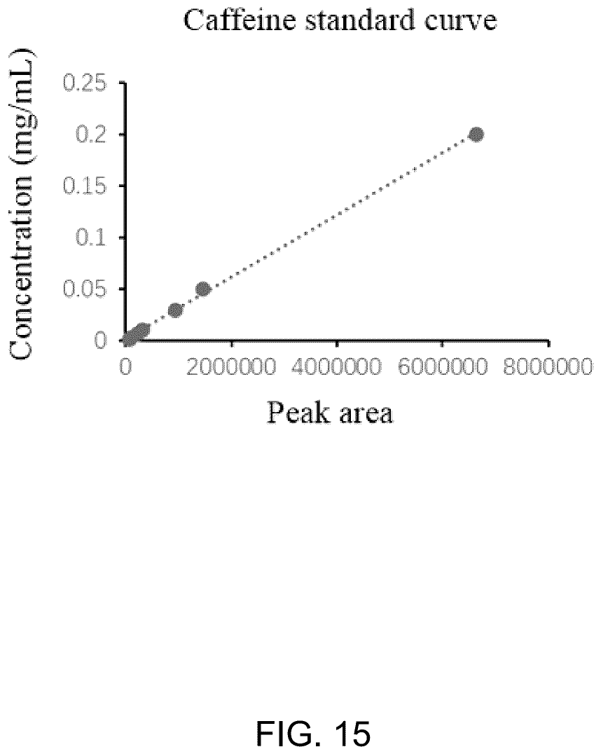

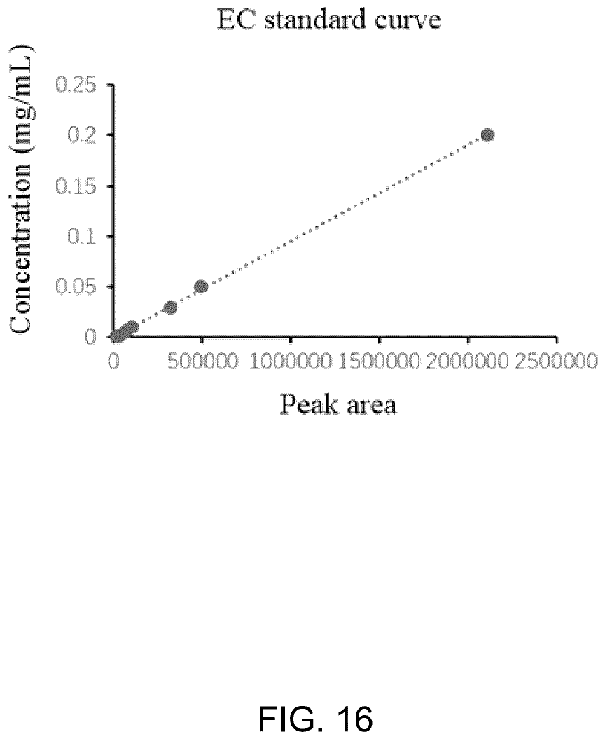

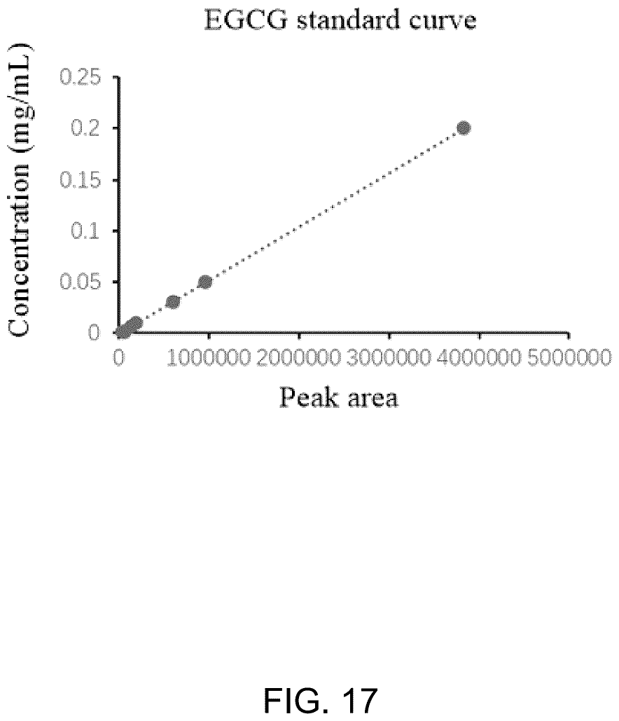

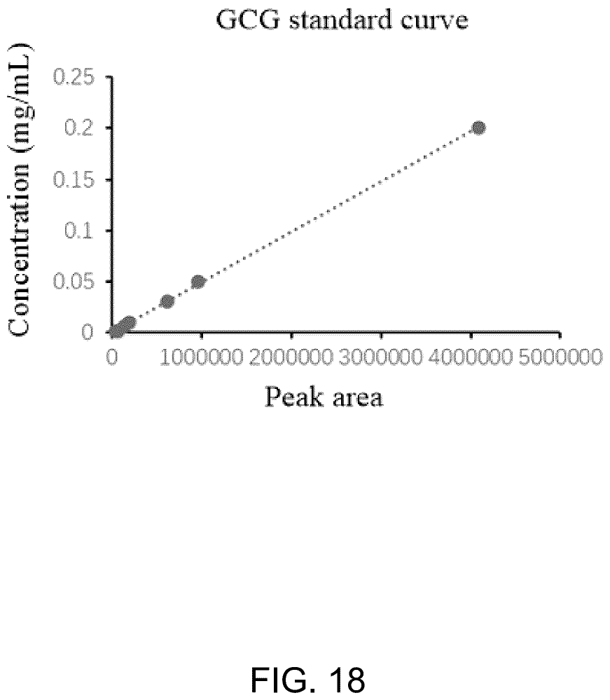

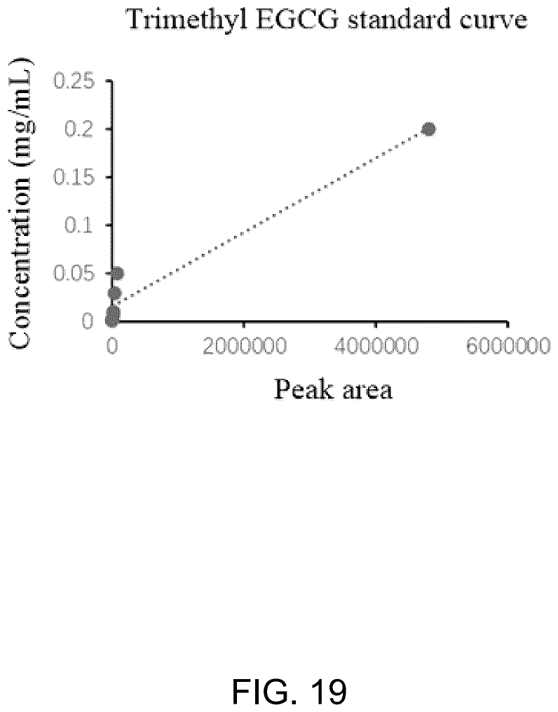

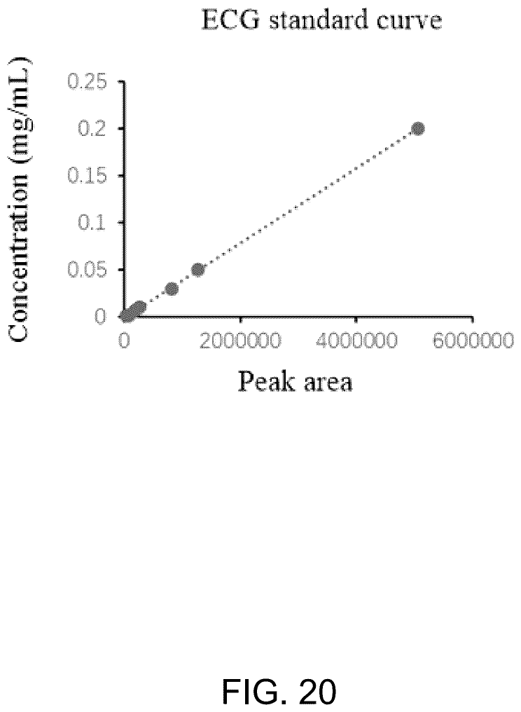

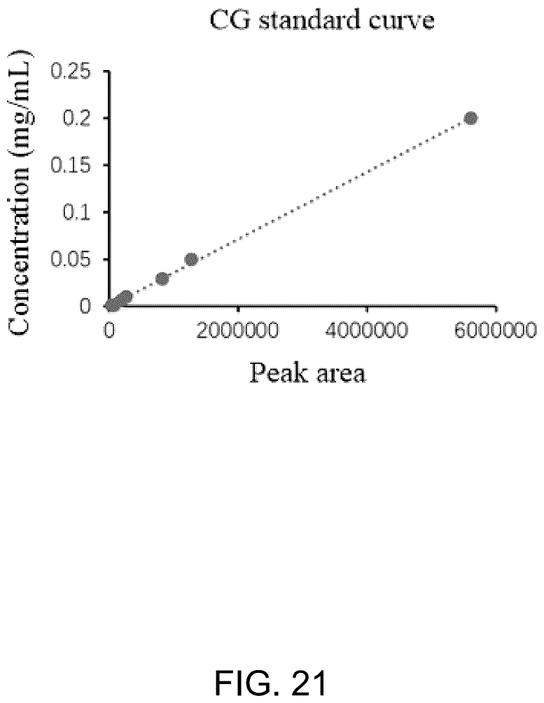

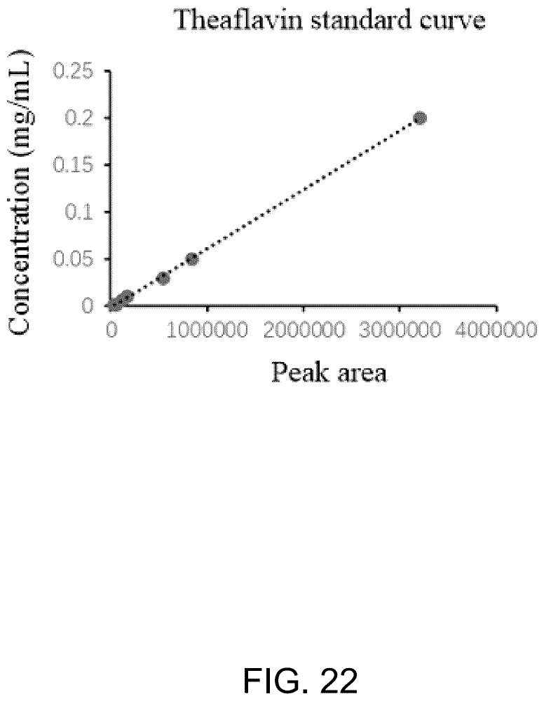

Plotting standard curves: accurately weighing 10 mg of standard samples of catechins and alkaloids respectively, placing the samples in volumetric flasks, dissolving the samples with a methanol solution, and making up to volume of 10 mL to prepare a standard stock solution with content of 1 mg/mL for each standard substance; and then diluting the standard stock solution as a mother solution with the methanol solution to prepare a standard solution with a series of gradient concentrations of 0.0012 mg/mL, 0.002 mg/mL, 0.006 mg/mL, 0.01 mg/mL, 0.03 mg/mL, 0.05 mg/mL, and 0.2 mg/mL respectively, and plotting standard curves. Quantifying each compound using the standard curves, where results of the standard curves are shown in Table 1 and FIGS. 9 to 22.

Then, using high-performance liquid chromatography (HPLC) to detect the standard solutions and the extracts obtained after tea leaf treatment, and calculating contents of the 14 compounds.

TABLE 1
Standard curve calculation formulas of 14 compounds

| Peak | | | R-squared |
|---|---|---|---|
| No. | Compound | Standard curve calculation formula | value |
| 1 | Theobromine | y = 0.0000007891x − 0.0166831324 | R2 = 0.8292 |
| 2 | GC | y = 0.0000004017x + 0.0010517384 | R2 = 0.9982 |
| 3 | Theophylline | y = 0.0000000375x + 0.0033199745 | R2 = 0.9952 |
| 4 | EGC | y = 0.0000005741x − 0.0051135036 | R2 = 0.9956 |
| 5 | Theacrine | y = 0.0000000566x − 0.0000166743 | R2 = 0.9996 |
| 6 | C | y = 0.0000001059x − 0.0004573687 | R2 = 0.9998 |
| 7 | Caffeine | y = 0.0000000301x + 0.0008798432 | R2 = 0.9989 |
| 8 | EC | y = 0.0000000952x − 0.0000938551 | R2 = 0.9996 |
| 9 | EGCG | y = 0.0000000524x − 0.0007481208 | R2 = 0.9999 |
| 10 | GCG | y = 0.0000000491x − 0.0000211166 | R2 = 0.9996 |
| 11 | EGCG3″Me | y = 0.0000000386x + 0.0155558812 | R2 = 0.9444 |
| 12 | ECG | y = 0.0000000398x − 0.0010347395 | R2 = 0.9999 |
| 13 | CG | y = 0.0000000357x + 0.0003487674 | R2 = 0.9994 |
| 14 | Theaflavin | y = 0.0000000627x − 0.0016788250 | R2 = 0.9997 |

### Example 1

A method for simultaneously detecting four alkaloids and 10 catechin components in tea includes the following steps:

10 mg of standard samples of catechins and alkaloids were accurately weighed respectively, the samples were placed in volumetric flasks, dissolved with a methanol solution, and made up to volume of 10 mL to prepare a standard stock solution with content of 1 mg/ml for each standard substance; then the standard stock solution was diluted with the methanol solution to prepare a standard solution with a series of gradient concentrations of 0.0012 mg/mL, 0.002 mg/mL, 0.006 mg/mL, 0.01 mg/mL, 0.03 mg/mL, 0.05 mg/mL, and 0.2 mg/mL respectively, standard curves were plotted, and each metabolite was quantified based on the standard curves; and

the content was determined by high-performance liquid chromatography, and chromatographic conditions were as follows: a chromatographic column was Agilent Zorbax Eclipse Plus C18 (4.6×250 mm, 5 μm), a column temperature was 35° C., a mobile phase C was 100% acetonitrile, a mobile phase D was a 0.05% phosphoric acid aqueous solution, a flow rate was 1 mL/min, a detection wavelength was 280 nm, and an injection volume was 10 μL; and quantification based on a peak area was performed according to an external standard method, with a gradient elution program shown in Table 2. The 0.05% phosphoric acid aqueous solution was prepared by the following method: 0.50 mL of phosphoric acid was pipetted, water was added to make up to volume in a 1,000 mL volumetric flask, and a resulting mixture was mixed well, where a pH value was 1.9.

TABLE 2
Liquid chromatography elution program

| Time (min) | Mobile phase C/% | Mobile phase D/% |
|---|---|---|
| 0.01 | 6.5 | 93.5 |
| 30 | 16 | 84 |
| 34 | 20 | 80 |
| 39 | 20 | 80 |
| 40 | 35 | 65 |
| 45 | 35 | 65 |
| 50 | 6.5 | 93.5 |
| 60 | End | End |

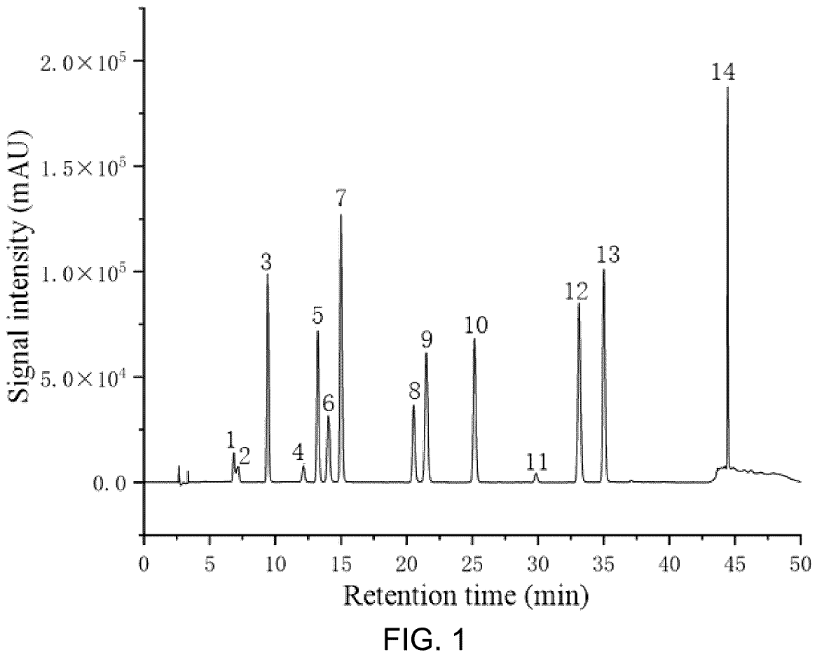

Detection results of standard samples of 14 compounds are shown in FIG. 1 and Table 3.

TABLE 3
Liquid chromatography information of standard
samples of four alkaloids and 10 catechins

| Peak | | Time | | Peak | Concentration |
|---|---|---|---|---|---|
| No. | Compound | (min) | Area | height | (mg/mL) |
| 1 | Theobromine | 6.833 | 146713 | 13847 | 0.2 |
| 2 | GC | 7.146 | 105789 | 7410 | 0.2 |
| 3 | Theophylline | 9.409 | 975207 | 98689 | 0.2 |
| 4 | EGC | 12.127 | 109081 | 7713 | 0.2 |
| 5 | Theacrine | 13.215 | 828691 | 71762 | 0.2 |
| 6 | C | 14.032 | 454287 | 31416 | 0.2 |
| 7 | Caffeine | 14.984 | 1463586 | 127036 | 0.2 |
| 8 | EC | 20.516 | 495478 | 36661 | 0.2 |
| 9 | EGCG | 21.477 | 953807 | 61362 | 0.2 |
| 10 | GCG | 25.163 | 959579 | 68403 | 0.2 |
| 11 | EGCG3″Me | 29.850 | 67198 | 4619 | 0.2 |
| 12 | ECG | 33.161 | 1262665 | 85162 | 0.2 |
| 13 | CG | 35.050 | 1285586 | 101152 | 0.2 |
| 14 | Theaflavin | 44.453 | 843966 | 181275 | 0.2 |

The repeatability, precision and stability of the above method were tested:
Repeatability test: three identical samples were taken, sample injection analysis thereof was performed respectively, peak areas of the four alkaloids and 10 catechins were recorded, and the content of the four alkaloids and 10 catechins in the sample was calculated by a standard curve method;
precision test: 10 μL of a sample solution was pipetted, sample injection was performed for four consecutive times, and peak areas of four alkaloids and 10 catechins were determined; and
stability test: a sample solution was pipetted, sample injection analysis was performed at 0 h, 2 h, 4 h, 8 h, 16 h, and 24 h after preparation respectively, and peak areas of four alkaloids and 10 catechins were recorded, to test the stability of the sample.

The results are shown in Table 4. The results show that relative standard deviation (RSD) values of each component range from 0.508% to 2.603%, indicating that the method demonstrates excellent repeatability under optimal gradient elution conditions. Results of the precision test show that the RSD values of each component range from 0.581% to 3.190%, indicating that the method has good precision. Results of the stability test show that the RSD values of each component range from 0.544% to 2.659%, indicating that the method has good stability.

TABLE 4
Results of repeatability, precision and stability
tests of four alkaloids and 10 catechins

| Serial | | RSD of | RSD of | RSD of |
|---|---|---|---|---|
| No. | Compound | repeatability (%) | precision (%) | stability (%) |
| 1 | Theobromine | 0.987 | 1.164 | 1.263 |
| 2 | GC | 0.508 | 1.624 | 1.663 |
| 3 | Theophylline | 0.633 | 0.652 | 0.544 |
| 4 | EGC | 2.082 | 1.392 | 2.219 |
| 5 | Theacrine | 2.603 | 3.190 | 2.659 |
| 6 | C | 1.147 | 0.581 | 1.142 |
| 7 | Caffeine | 0.752 | 0.954 | 0.841 |
| 8 | EC | 1.381 | 1.642 | 1.600 |
| 9 | EGCG | 0.987 | 1.089 | 1.064 |
| 10 | GCG | 1.855 | 2.681 | 2.492 |
| 11 | EGCG3″Me | 1.166 | 0.585 | 0.927 |
| 12 | ECG | 0.701 | 1.044 | 1.136 |
| 13 | CG | 1.435 | 1.414 | 2.500 |
| 14 | Theaflavin | 0.902 | 1.442 | 1.373 |

The above method was used to detect 14 compounds in black tea, with specific steps as follows:

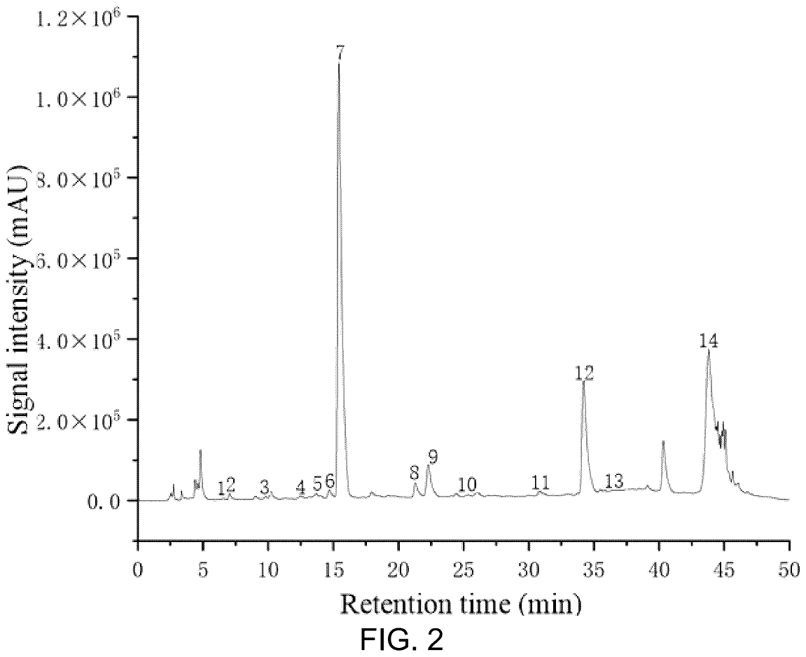

0.2 g (accurate to 0.0001 g) of a uniformly ground black tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 2 and Table 5.

TABLE 5
Chromatographic information of four alkaloids
and 10 catechins in black tea

| Peak | | Time | | Peak | Concentration |
|---|---|---|---|---|---|
| No. | Compound | (min) | Area | height | (mg/mL) |
| 1 | Theobromine | 6.572 | 65884 | 2625 | 1.080 |
| 2 | GC | 7.042 | 212144 | 15053 | 4.313 |
| 3 | Theophylline | 9.805 | 93421 | 5922 | 0.341 |
| 4 | EGC | 12.454 | 182954 | 6431 | 4.996 |
| 5 | Theacrine | 13.697 | 178528 | 9809 | 0.504 |
| 6 | C | 14.697 | 433079 | 21520 | 2.270 |
| 7 | Caffeine | 15.436 | 24603117 | 1079343 | 37.072 |
| 8 | EC | 21.303 | 858201 | 36945 | 4.080 |
| 9 | EGCG | 22.301 | 2017726 | 81555 | 5.249 |
| 10 | GCG | 26.075 | 259660 | 9587 | 0.636 |
| 11 | EGCG3″Me | 30.835 | 343137 | 12391 | 12.840 |
| 12 | ECG | 34.217 | 7418915 | 283375 | 14.712 |
| 13 | CG | 36.261 | 8247 | 939 | 0.032 |
| 14 | Theaflavin | 44.675 | 516010 | 34302 | 1.533 |

### Example 2

The difference between Example 2 and Example 1 lies in that yellow tea was detected in Example 2.

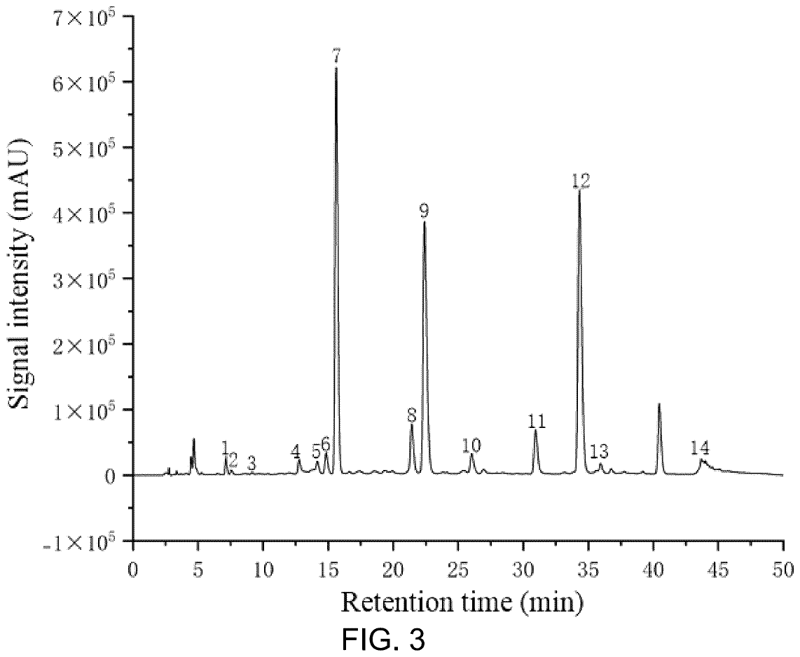

0.2 g (accurate to 0.0001 g) of a uniformly ground yellow tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 3 and Table 6.

TABLE 6
Chromatographic information of four alkaloids
and 10 catechins in yellow tea

| Peak | | Time | | Peak | Concentration |
|---|---|---|---|---|---|
| No. | Compound | (min) | Area | height | (mg/mL) |
| 1 | Theobromine | 7.142 | 252363 | 24748 | 4.298 |
| 2 | GC | 7.568 | 88360 | 7398 | 1.827 |
| 3 | Theophylline | 9.933 | 5368 | 403 | 0.176 |
| 4 | EGC | 13.87 | 13883 | 1556 | 0.143 |
| 5 | Theacrine | 14.173 | 290174 | 19103 | 0.820 |
| 6 | C | 14.845 | 486333 | 31900 | 2.552 |
| 7 | Caffeine | 15.621 | 9442840 | 619130 | 14.255 |
| 8 | EC | 21.428 | 1375821 | 75774 | 6.544 |
| 9 | EGCG | 22.415 | 7640311 | 384564 | 19.980 |
| 10 | GCG | 26.04 | 584865 | 30817 | 1.435 |
| 11 | EGCG3″Me | 30.95 | 1324000 | 67917 | 49.677 |
| 12 | ECG | 34.326 | 8770003 | 431036 | 17.401 |
| 13 | CG | 35.953 | 239282 | 15220 | 0.445 |
| 14 | Theaflavin | 44.551 | 31823 | 4276 | 0.016 |

### Example 3

The difference between Example 3 and Example 1 lies in that green tea was detected in Example 3.

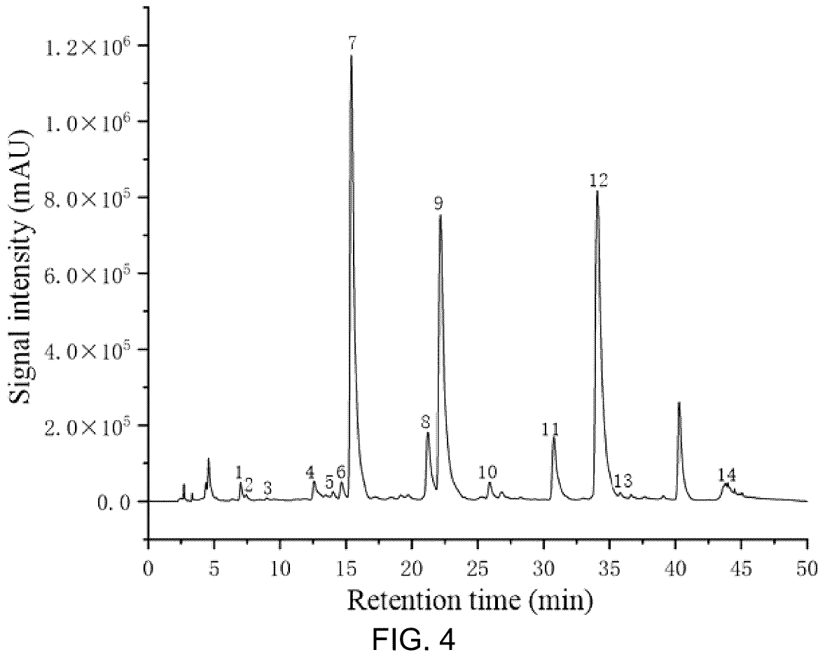

0.2 g (accurate to 0.0001 g) of a uniformly ground green tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 4 and Table 7.

TABLE 7
Chromatographic information of four alkaloids
and 10 catechins in green tea

| Peak | | Time | Peak | Peak | Concentration |
|---|---|---|---|---|---|
| No. | Compound | (min) | area | height | (mg/mL) |
| 1 | Theobromine | 7.019 | 593623 | 47939 | 10.188 |
| 2 | GC | 7.423 | 245290 | 14653 | 4.979 |
| 3 | Theophylline | 9.748 | 21836 | 1662 | 0.207 |
| 4 | EGC | 12.592 | 1224226 | 49205 | 34.886 |
| 5 | Theacrine | 13.476 | 53230 | 4604 | 0.150 |
| 6 | C | 14.672 | 818426 | 44157 | 4.311 |
| 7 | Caffeine | 15.415 | 28290086 | 1166753 | 42.621 |
| 8 | EC | 21.217 | 4083386 | 169637 | 19.432 |
| 9 | EGCG | 22.182 | 21854112 | 737539 | 57.220 |
| 10 | GCG | 25.904 | 772148 | 41731 | 1.895 |
| 11 | EGCG3″Me | 30.778 | 4470452 | 166504 | 167.842 |
| 12 | ECG | 34.082 | 26156035 | 804371 | 51.999 |
| 13 | CG | 35.808 | 200209 | 13529 | 0.375 |
| 14 | Theaflavin | 44.496 | 92933 | 14985 | 0.207 |

### Example 4

The difference between Example 4 and Example 1 lies in that dark tea was detected in Example 4.

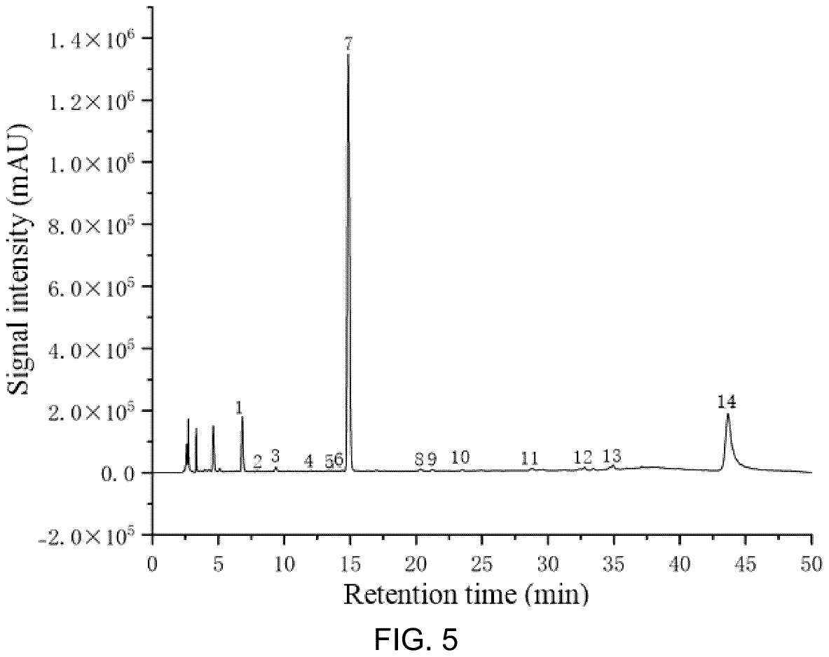

0.2 g (accurate to 0.0001 g) of a uniformly ground dark tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 5 and Table 8.

TABLE 8
Chromatographic information of four alkaloids
and 10 catechins in dark tea

| | | Retention | | | |
|---|---|---|---|---|---|
| Peak | | time | Peak | Peak | Concentration |
| No. | Compound | (min) | area | height | (mg/mL) |
| 1 | Theobromine | 6.824 | 1487069 | 177154 | 25.609 |
| 2 | GC | 7.088 | 4691 | 1494 | 0.147 |
| 3 | Theophylline | 9.371 | 122780 | 14059 | 0.396 |
| 4 | EGC | 12.027 | 27234 | 2402 | 0.526 |
| 5 | Theacrine | 13.245 | 19551 | 1485 | 0.054 |
| 6 | C | 13.866 | 13054 | 1263 | 0.046 |
| 7 | Caffeine | 14.859 | 16406466 | 1343162 | 24.736 |
| 8 | EC | 20.327 | 94525 | 6482 | 0.445 |
| 9 | EGCG | 21.237 | 75300 | 5164 | 0.160 |
| 10 | GCG | 24.916 | 21491 | 1411 | 0.052 |
| 11 | EGCG3″Me | 29.635 | 46308 | 3209 | 1.693 |
| 12 | ECG | 33.438 | 93929 | 6521 | 0.135 |
| 13 | CG | 34.946 | 157092 | 12771 | 0.298 |
| 14 | Theaflavin | 43.665 | 459025 | 182194 | 1.355 |

### Example 5

The difference between Example 5 and Example 1 lies in that white tea was detected in Example 5.

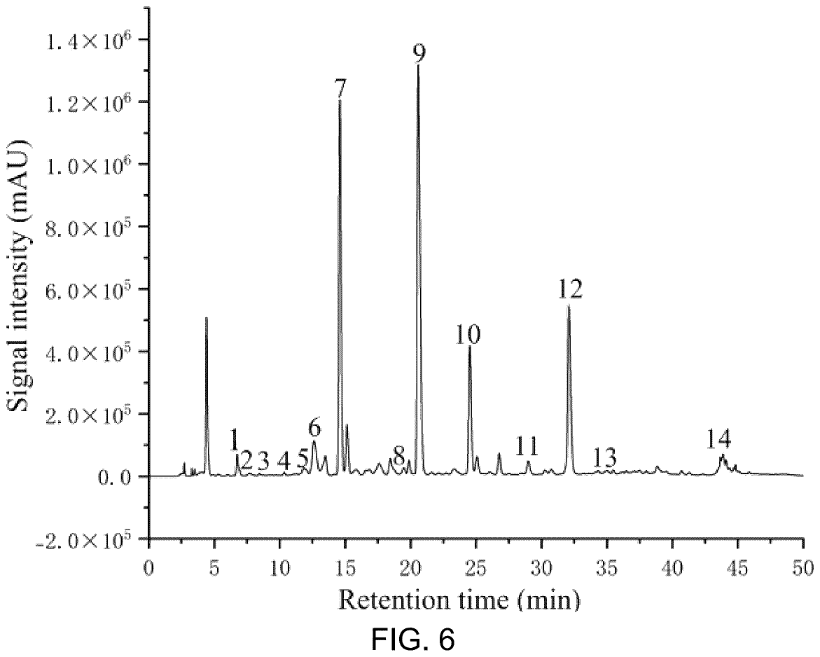

0.2 g (accurate to 0.0001 g) of a uniformly ground white tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 6 and Table 9.

TABLE 9
Chromatographic information of four alkaloids
and 10 catechins in white tea

| | | Retention | | | |
|---|---|---|---|---|---|
| Peak | | time | Peak | Peak | Concentration |
| No. | Compound | (min) | area | height | (mg/mL) |
| 1 | Theobromine | 6.755 | 581565 | 68489 | 9.980 |
| 2 | GC | 6.896 | 129138 | 20622 | 2.646 |
| 3 | Theophylline | 9.189 | 11037 | 1084 | 0.187 |
| 4 | EGC | 11.842 | 370783 | 18073 | 10.388 |
| 5 | Theacrine | 12.622 | 2274151 | 103639 | 6.435 |
| 6 | C | 13.500 | 834986 | 62627 | 4.398 |
| 7 | Caffeine | 14.596 | 14257746 | 1201296 | 21.502 |
| 8 | EC | 19.882 | 522899 | 44479 | 2.484 |
| 9 | EGCG | 20.589 | 20929394 | 1323478 | 54.798 |
| 10 | GCG | 24.531 | 5585773 | 410114 | 13.712 |
| 11 | EGCG3″Me | 28.994 | 699126 | 43183 | 26.209 |
| 12 | ECG | 32.107 | 8850261 | 551570 | 17.560 |
| 13 | CG | 34.332 | 94118 | 8265 | 0.185 |
| 14 | Theaflavin | 44.357 | 107346 | 12064 | 0.253 |

### Example 6

The difference between Example 6 and Example 1 lies in that oolong tea was detected in Example 6.

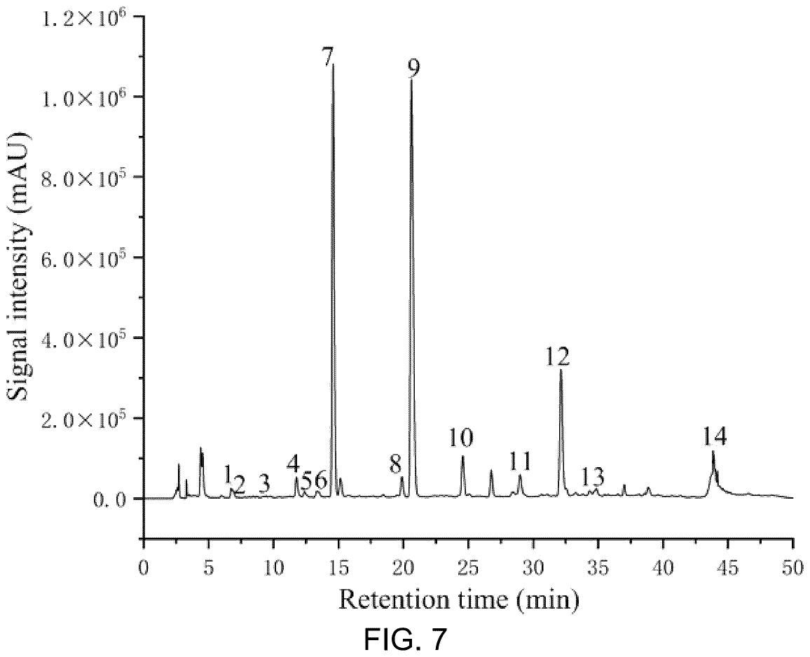

0.2 g (accurate to 0.0001 g) of a uniformly ground oolong tea sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 7 and Table 10.

TABLE 10
Chromatographic information of four alkaloids
and 10 catechins in oolong tea

| | | Retention | | | |
|---|---|---|---|---|---|
| Peak | | time | Peak | Peak | Concentration |
| No. | Compound | (min) | area | height | (mg/mL) |
| 1 | Theobromine | 6.754 | 214417 | 22726 | 3.643 |
| 2 | GC | 6.896 | 121984 | 16408 | 2.503 |
| 3 | Theophylline | 9.198 | 33006 | 2555 | 0.228 |
| 4 | EGC | 11.771 | 609312 | 49141 | 17.235 |
| 5 | Theacrine | 12.385 | 183052 | 13235 | 0.517 |
| 6 | C | 13.347 | 177228 | 15500 | 0.916 |
| 7 | Caffeine | 14.594 | 12608520 | 1077325 | 19.020 |
| 8 | EC | 19.891 | 664254 | 51107 | 3.157 |
| 9 | EGCG | 20.626 | 15916378 | 1038856 | 41.664 |
| 10 | GCG | 24.586 | 1291560 | 99349 | 3.170 |
| 11 | EGCG3″Me | 28.992 | 685617 | 48491 | 25.702 |
| 12 | ECG | 32.139 | 4811265 | 315960 | 9.523 |
| 13 | CG | 34.347 | 182905 | 11994 | 0.344 |
| 14 | Theaflavin | 44.182 | 181762 | 39346 | 0.486 |

### Example 7

The difference between Example 7 and Example 1 lies in that fresh tea leaves were detected in Example 7.

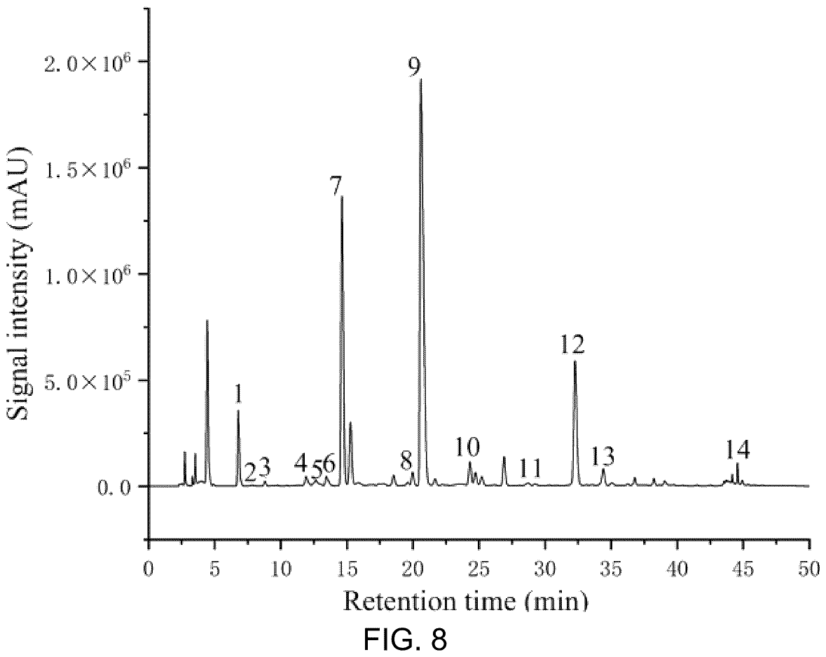

0.2 g (accurate to 0.0001 g) of a uniformly ground fresh tea leaf sample was weighed into a 10 mL centrifuge tube, 5 mL of a 70% methanol aqueous solution preheated in a 70° C. water bath was added, a resulting mixture was fully mixed, immediately transferred to the 70° C. water bath for extraction for 10 min, shaken every 5 min, cooled to room temperature after the extraction, and centrifuged at 3,500 r/min for 10 min, and a supernatant was transferred to a 10 mL volumetric flask. Residues were extracted once with 5 mL of a 70% methanol aqueous solution, the above operation was repeated, extracts were combined, made up to volume of 10 mL, and shaken well, and filtering with a 0.45 μm filter membrane was performed for liquid chromatography analysis, with the results shown in FIG. 8 and Table 11.

TABLE 11
Chromatographic information of four alkaloids
and 10 catechins in fresh tea leaves

| | | Retention | | | |
|---|---|---|---|---|---|
| Peak | | time | Peak | Peak | Concentration |
| No. | Compound | (min) | area | height | (mg/mL) |
| 1 | Theobromine | 6.79 | 3263902 | 356763 | 56.277 |
| 2 | GC | 7.831 | 79845 | 3294 | 1.656 |
| 3 | Theophylline | 8.804 | 221860 | 21938 | 0.582 |
| 4 | EGC | 11.916 | 627968 | 42718 | 17.770 |
| 5 | Theacrine | 12.651 | 499055 | 23232 | 1.411 |
| 6 | C | 13.452 | 639177 | 43204 | 3.362 |
| 7 | Caffeine | 14.636 | 16558312 | 1361844 | 24.964 |
| 8 | EC | 19.976 | 786047 | 64299 | 3.737 |
| 9 | EGCG | 20.613 | 35469217 | 1913660 | 92.892 |
| 10 | GCG | 24.731 | 714589 | 57039 | 1.753 |
| 11 | EGCG3″Me | 29.25 | 153049 | 8515 | 5.701 |
| 12 | ECG | 32.272 | 9195351 | 586300 | 18.247 |
| 13 | CG | 34.413 | 1165866 | 78963 | 2.099 |
| 14 | Theaflavin | 44.558 | 54445 | 102708 | 0.086 |

The above are merely some embodiments of the present disclosure. For those of ordinary skill in the art, they may also make several transformations and improvements on the premise of not deviating from the inventive concept of the present disclosure, and these transformations and improvements shall fall within the scope of protection of the present disclosure.

What is claimed is:

1. A method for simultaneously detecting four alkaloids and 10 catechin components in tea, comprising the following steps:
simultaneously detecting four alkaloids and 10 catechin components in a tea sample by using high-performance liquid chromatography, and obtaining content of the 14 characteristic components in the tea sample according to respective standard curves of the four alkaloids and 10 catechin components; chromatographic conditions are as follows: a chromatographic column is Agilent Zorbax Eclipse Plus C18, a column temperature is 30-40° C., a mobile phase C is 100% acetonitrile, a mobile phase D is a 0.05% phosphoric acid aqueous solution, a flow rate is 0.9-1.1 mL/min, and a detection wavelength is 275-285 nm; and performing quantification based on a peak area according to an external standard method, wherein a gradient elution program is as follows:

| Time/min | Mobile phase C/% | Mobile phase D/% |
|---|---|---|
| 0.01 | 6.5 | 93.5 |
| 30 | 16 | 84 |
| 34 | 20 | 80 |
| 39 | 20 | 80 |
| 40 | 35 | 65 |
| 45 | 35 | 65 |
| 50 | 6.5 | 93.5 |
| 60 | End | End |

wherein the four alkaloids are theophylline, theobromine, caffeine, and theacrine; and the 10 catechins are epicatechin, catechin, epigallocatechin, epicatechin gallate, epigallocatechin gallate, catechin gallate, gallocatechin gallate, gallocatechin, epigallocatechin-3-O-(3-O-methyl) gallate, and theaflavin.

2. The method for simultaneously detecting four alkaloids and 10 catechin components in tea according to claim 1, wherein the standard curves are obtained by the following method: weighing standard samples of catechins and alkaloids respectively, dissolving the samples with a methanol solution, and making up to volume to prepare a standard stock solution with content of 1 mg/mL for each standard substance; and then diluting the standard stock solution with the methanol solution to prepare a standard solution with a series of gradient concentrations of 0.0012 mg/mL, 0.002 mg/mL, 0.006 mg/mL, 0.01 mg/mL, 0.03 mg/mL, 0.05 mg/mL, and 0.2 mg/mL respectively, performing detection according to the chromatographic conditions to obtain corresponding chromatograms, and plotting standard curves with concentration as the abscissa and peak area as the ordinate.

3. The method for simultaneously detecting four alkaloids and 10 catechin components in tea according to claim 1, wherein the tea sample is obtained by the following treatment method: after grinding tea, adding a 70% methanol aqueous solution preheated in a water bath, fully mixing, immediately transferring a resulting mixture to the water bath for extraction, cooling to room temperature after the extraction, and centrifuging to collect a supernatant; and extracting centrifuged residues once with the 70% methanol aqueous solution, repeating the above operation, combining extracts and making up to volume, shaking well, and filtering.

4. The method for simultaneously detecting four alkaloids and 10 catechin components in tea according to claim 2, wherein the tea sample is obtained by the following treatment method: after grinding tea, adding a 70% methanol aqueous solution preheated in a water bath, fully mixing, immediately transferring a resulting mixture to the water bath for extraction, cooling to room temperature after the extraction, and centrifuging to collect a supernatant; and extracting centrifuged residues once with the 70% methanol aqueous solution, repeating the above operation, combining extracts and making up to volume, shaking well, and filtering.

5. The method for simultaneously detecting four alkaloids and 10 catechin components in tea according to claim 3, wherein the tea sample is obtained by the following treatment method: weighing 0.2 g of uniformly ground tea into a 10 mL centrifuge tube, adding 5 mL of a 70% methanol aqueous solution preheated in a 60-80° C. water bath, fully mixing, immediately transferring a resulting mixture to the 60-80° C. water bath for extraction for 8-12 min, shaking every 3-6 min, cooling to room temperature after extraction, centrifuging at 3,000-4,000 r/min for 8-12 min, and transferring a supernatant to a 10 mL volumetric flask; and extracting residues once with 5 mL of a 70% methanol aqueous solution, repeating the above operation, combining extracts and making up to volume of 10 mL, and filtering with a 0.45 μm filter membrane.

6. The method for simultaneously detecting four alkaloids and 10 catechin components in tea according to claim 3, wherein the tea sample is obtained by the following treatment method: weighing 0.2 g of uniformly ground tea into a 10 mL centrifuge tube, adding 5 mL of a 70% methanol aqueous solution preheated in a 70° C. water bath, mixing and immediately transferring a resulting mixture to the 70° C. water bath for extraction for 10 min, shaking every 5 min, cooling to room temperature after the extraction, centrifuging at 3,500 r/min for 10 min, and transferring a supernatant to a 10 mL volumetric flask; and extracting residues once with 5 mL of a 70% methanol aqueous solution, repeating the above operation, combining extracts and making up to volume of 10 mL, mixing, and filtering with a 0.45 μm filter membrane.

7. The method for simultaneously detecting four alkaloids and 10 catechin components in tea according to claim 1, wherein the 0.05% phosphoric acid aqueous solution is prepared by the following method: pipetting 0.50 mL of phosphoric acid, adding water and making up to volume in a 1,000 mL volumetric flask, and mixing, wherein a pH value is 1.8-2.0.

8. The method for simultaneously detecting four alkaloids and 10 catechin components in tea according to claim 1, wherein the flow rate is 1 mL/min.

9. The method for simultaneously detecting four alkaloids and 10 catechin components in tea according to claim 1, wherein the column temperature is 35° C.

10. The method for simultaneously detecting four alkaloids and 10 catechin components in tea according to claim 1, wherein the detection wavelength is 280 nm.

11. The method for simultaneously detecting four alkaloids and 10 catechin components in tea according to claim 1, wherein an injection volume is 10 μL.
